(19) United States
(12) Patent Application Publication
(10) Pub. No.: US 2012/0165956 A1
(43) Pub. Date: Jun. 28, 2012

(54) UROLOGICAL MEDICAL DEVICES

(75) Inventor: Jianmin Li, Lexington, MA (US)

(73) Assignee: Boston Scientific Scimed, Inc., Maple Grove, MN (US)

(21) Appl. No.: 13/328,536

(22) Filed: Dec. 16, 2011

Related U.S. Application Data
(60) Provisional application No. 61/426,299, filed on Dec. 22, 2010.

(51) Int. Cl. A61F 2/04; A61P 13/02; A61K 31/728

(52) U.S. Cl. 623/23.66; 514/54; 424/430

(57) ABSTRACT

According to an aspect of the present invention, urological medical devices are provided which comprise a substrate and hyaluronic acid, which is releasably disposed on or in the substrate.

### RELATED APPLICATION SECTION

This application claims the benefit of U.S. Provisional Patent Application Ser. No. 61/426,299, filed Dec. 22, 2010, entitled “UROLOGICAL MEDICAL DEVICES,” which is incorporated by reference herein in its entirety.

### FIELD OF THE INVENTION

The present invention relates generally to medical devices, and more particularly to urological medical devices that release hyaluronic acid.

### BACKGROUND OF THE INVENTION

Urological medical devices such as urinary “Foley” catheters, urethral stents and ureteral stents, among others, have been useful for treating a variety of medical conditions. A drawback of such medical devices, however, is the risk of infection that arises as a result of the implantation/insertion of the medical device in the urinary tract. Such risk exists even though the medical devices are sterilized and carefully packaged to guard against introduction of microbes or pathogens.

When catheters and other in-dwelling urological devices are implanted or inserted into the urinary tract, bacteria or other microorganisms can be picked up and carried into the implantation/insertion site where microbial colonization may ensue. Infections may derive from an interaction of the microorganisms and the device surface. Once infected, microorganisms may adhere to the device surface and rapidly become encased in a polysaccharide matrix or biofilm, which protects the microorganisms from a host's defenses. Infections may also derive from an interaction of microorganisms and the host's urinary tract, which can lead to urinary tract infections (UTI). One hypothesis is that microbial infections of the urinary tract may occur as a result of pre-existing or surgery-related irregularities which makes it easy for microorganisms to settle.

Regardless of the mechanisms of infection, there is a need for the development of implantable/insertable devices that are capable of preventing, reducing and/or treating infections caused by the implantation or insertion of such devices into a patient's body.

### SUMMARY OF THE INVENTION

According to an aspect of the present invention, urological medical devices are provided which comprise a substrate and hyaluronic acid, which is releasably disposed on or in the substrate.

Advantages of the invention include one or more of the following: (a) devices are provided which release hyaluronic acid into the urinary tract in quantities sufficient to prevent, reduce and/or eliminate local microbial infection in the urinary tract (e.g., microbial colonization of host tissue and/or the device itself); and (b) devices are provided which release hyaluronic acid into the urinary tract in quantities sufficient to coat portions of the urinary tract that are adjacent to and downstream of such devices, including damaged portions of the urinary tract (e.g., imperfect epithelial membrane) where present.

These and other aspects, embodiments and advantages of the present invention will become immediately apparent to those of ordinary skill in the art upon review of the Detailed Description and any claims to follow.

### BRIEF DESCRIPTION OF THE DRAWINGS

FIG. 1 is a schematic side view of a ureteral stent, in accordance with an embodiment of the invention.

FIGS. 2A-2D are schematic cross-sectional views of the ureteral stent, taken along plane ii-ii, in accordance with various embodiments of the invention.

FIG. 3 is a schematic partial cross-sectional view of a ureteral stent, in accordance with an embodiment of the invention.

FIG. 4A is a schematic end view of a ureteral stent in accordance with an embodiment of the invention. FIG. 4B is a schematic partial cross-sectional view of the stent of FIG. 4A, taken along plane A-A. FIG. 4C is an expanded view of FIG. 4B corresponding to region B of FIG. 4B.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

The medical devices of the present invention are urological medical devices, including any medical device that is suitable for placement in the urinary tract of a subject, including the kidneys (e.g., in the renal calyx, renal pelvis, etc.), ureters, bladder and urethra.

According to an aspect of the present invention, urological medical devices are provided which comprise a substrate and hyaluronic acid, which is releasably disposed in or on the substrate. Examples of such substrates include urinary (e.g., Foley) catheters and portions thereof, ureteral stents and portions thereof, urethral stents and portions thereof, ureteral catheters and portions thereof, and so forth.

In various embodiments, upon implantation or insertion of such a device in a host's urinary tract, the medical device releases the hyaluronic acid (a) in an amount effective to coat at least portions of the urinary tract that are adjacent to and downstream of such devices (e.g., urethra; urinary bladder and urethra; ureter, urinary bladder and urethra; kidney, ureter, urinary bladder and urethra), including damaged portions of the urinary tract (e.g., imperfect epithelial membrane) where present and/or (b) in an amount effective to prevent, reduce and/or eliminate local microbial infection in the urinary tract (relative to the implantation/insertion of the device in the absence of the hyaluronic acid release).

Without wishing to be bound by theory, it is believed that, by releasing hyaluronic acid into the urinary tract, that devices of the present invention disclosure are effective to coat portions of the urinary tract that lie adjacent to and downstream of the devices, thereby forming a physical barrier for the prevention of microbial attachment and/or colonization, such that local infection is reduced or eliminated. Furthermore, decreased microbial presence in the urinary tract reduces the likelihood that biofilms will form on the medical devices of the present invention, thereby reducing or eliminating encrustation formation.

In certain embodiments, the existence of hyaluronic acid exhibits a reduction in microbial infection (e.g., due to bacteria or other common urinary tract microorganisms, such as Escherichia coli, and Streptococcus aureus), relative to the same medium in the absence of the hyaluronic acid. Clinical studies have demonstrated intravesical instillation of hyaluronic acid results in reduction or elimination of recurrent of urinary track infection for at least 3 months.

Subjects for the devices of the present invention (also referred to as patients, hosts, etc.) include vertebrate subjects, particularly humans and various warm-blooded animals including pets (e.g., dogs, cats, etc.) and livestock (horses, cattle, etc.).

As noted above, urological medical devices are described herein which comprise a substrate and hyaluronic acid, which is releasably disposed in or on the substrate.

Examples of substrates include substrates that are formed from polymeric materials, ceramic materials, metallic materials, and combinations of the same.

In certain embodiments, polymers are preferred substrate materials. Polymers for forming medical device substrates may be selected from suitable members of the following, among others: polycarboxylic acid polymers and copolymers including polyacrylic acids; acetal polymers and copolymers; acrylate and methacrylate polymers and copolymers (e.g., n-butyl methacrylate); cellulosic polymers and copolymers; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; polycarbonates; polyacrylonitriles; polyvinylpyrrolidones; polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropenes) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers; as well as blends and further copolymers of the above.

Hyaluronic acid releasing compositions (for disposition on or in a medical device substrate) may be provided, for example, in a liquid form, semi-liquid form (e.g., gel form, etc.) or solid form.

Hyaluronic acid releasing compositions for use herein may be provided in solid form, for example, in the form of layers (e.g., ranging from 1 um to 100 um in thickness, and coating over or a portion of the substrate), in the form of regular and irregular particles (e.g., ranging from 0.05 μm to 0.1 μm to 0.5 μm to 1 μm to 5 μm to 10 μm to 50 μm in largest cross-sectional dimension, including powders, beads, pellets and tablets), in the form of rods (e.g., “drug sticks” ranging from 100 μm to 250 μm to 500 μm to 1000 μm to 2500 μm to 5000 μm to 10000 μm in length and 20 μm to 50 μm to 100 um and width and being of circular, oval, polygonal, etc. cross-section), and so forth.

Solid hyaluronic acid releasing compositions include substantially pure hyaluronic acid (i.e., at least 95 wt % to 97 wt % to 99 wt % or more hyaluronic acid).

Solid hyaluronic acid releasing compositions include compositions comprising hyaluronic acid hyaluronic acid (e.g., 0.05 wt % to 0.5 wt % to 1 wt % to 2 wt % to 5 wt % to 10 wt % to 25 wt % to 50 wt % to 75 wt % to 90 wt % to 95 wt % to 99 wt % to 99.5 wt % or more) combined with one or more optional supplemental agents (e.g., 99.95 wt % to 99.5 wt % to 99 wt % to 90 wt % to 75 wt % to 50 wt % to 25 wt % to 10 wt % to 5 wt % to 1 wt % to 0.5 wt % to 0.05 wt % or less).

Hyaluronic acid releasing compositions for use herein may also be provided in liquid or semi-liquid form, commonly in the form of layer a viscous liquid or gel. Such liquid or semi-liquid compositions are commonly employed as layers or reservoir filling materials. In addition to hyaluronic acid and a liquid component (e.g., water, other liquids such as saline and various buffers and combinations thereof), such liquid and semi-liquid compositions may also include one or more optional supplemental agents.

Non-liquid components of such liquid and semi-liquid compositions include substantially pure hyaluronic acid (i.e., at least 95 wt % to 97 wt % to 99 wt % or more hyaluronic acid).

Non-liquid components of such liquid and semi-liquid compositions may comprise hyaluronic acid (e.g., 0.05 wt % to 0.5 wt % to 1 wt % to 2 wt % to 5 wt % to 10 wt % to 25 wt % to 50 wt % to 75 wt % to 90 wt % to 95 wt % to 99 wt % to 99.5 wt % or more) and one or more optional supplemental agents (e.g., 99.95 wt % to 99.5 wt % to 99 wt % to 90 wt % to 75 wt % to 50 wt % to 25 wt % to 10 wt % to 5 wt % to 1 wt % to 0.5 wt % to 0.05 wt % or less)

The concentration of non-liquid hyaluronic components in such liquid or semi-liquid compositions typically ranges from 0.01 wt % to 0.02 wt % to 0.05 wt % to 1 wt % to 2 wt % to 5 wt % to 10 wt %.

Examples of optional supplemental agents include blending agents, therapeutic agents, and imaging agents, among others.

Examples of blending agents include suitable members of the following, among others: sugars, including sucrose, dextrose and so forth, polysaccharides including celluloses, for example, ionic celluloses such as sodium carboxymethyl cellulose, and non-ionic celluloses, for example, hydroxyalkyl celluloses such as hydroxymethyl cellulose, hydroxyethyl cellulose, and hydroxyproyl cellulose (e.g., Klucel® G and Klucel® E), further polysaccharides including alginic acid, pectinic acid, dextran, carboxymethyl dextran, modified dextran, starch, carboxymethyl starch, and additional polymers including polyethylene glycol, polyethylene terephthalate glycol (PETG), polyalkylene oxides including polyethylene oxide and polypropylene oxide, poly(acrylic acid), poly(methacrylic acid), polyvinyl alcohol, polyvinylpyrrolidone, polyacrylamide, poly(N-alkylacrylamides), poly(vinyl sulfonic acid), polyester amides, polyanhydrides, polyorthoesters, polyesters such as poly(lactide), poly(glycolide) and poly(lactide-co-glycolide), polyphosphazenes, poly(methyl methacrylate), poly(caprolactone), poly(dioxanone), poly(trimethylene carbonate), poly(methylene-bisacrylamide), proteins and polypeptides (e.g., polyglutamic acid, polylysine, etc.), suitable polymers from the above list of polymers for forming medical device substrates, as well as salts, copolymers and blends of the forgoing.

In some embodiments, soluble or biodegradable blending agents are preferred.

Examples of therapeutic agents may include, for example, corticosteroids, narcotic and non-narcotic analgesics, local anesthetic agents, antibiotics, anti-inflammation agents and combinations thereof, among others.

Examples of imaging agents include (a) contrast agents for use in connection with x-ray fluoroscopy, including metals, metal salts and oxides (particularly bismuth salts and oxides), and iodinated compounds, among others, (b) contrast agents for use in conjunction with ultrasound imaging, including inorganic and organic echogenic particles (i.e., particles that result in an increase in the reflected ultrasonic energy) or inorganic and organic echolucent particles (i.e., particles that result in a decrease in the reflected ultrasonic energy), and (c) contrast agents for use in conjunction with magnetic resonance imaging (MRI), including contrast agents that contain elements with relatively large magnetic moment such as Gd(III), Mn(II), Fe(III) and compounds (including chelates) containing the same, such as gadolinium ion chelated with diethylenetriaminepentaacetic acid.

In some embodiments, the hyaluronic acid within the hyaluronic acid releasing compositions may be ionically crosslinked or covalently crosslinked (e.g., via biodegradable bonds, such as ester bonds formed from the hydroxyl and carboxyl groups). In some embodiments, the hyaluronic acid within the hyaluronic acid releasing compositions is neither ionically nor covalently crosslinked. In some embodiments, a portion of the hyaluronic acid is un-crosslinked for more immediate release and a portion of the hyaluronic acid is ionically or covalently crosslinked for more delayed release.

Hyaluronic acid, as is well known, is an anionic, nonsulfated glycosaminoglycan. The hyaluronic acid employed in the devices of the invention may vary widely in molecular weight, for example, having a molecular weight ranges extending from 5,000 Da to 10,000 Da to 25,000 Da to 50,000 Da to 100,000 Da to 250,000 Da to 500,000 Da to 750,000 Da to 1,000,000 Da to 1,250,000 Da to 1,500,000 Da to 2,000,000 Da to 2,500,000 Da to 5,000,000 Da to 7,500,000 Da to 10,000,000 Da to 15,000,000 Da to 20,000,000 Da.

Unless otherwise indicated, “average molecular weight” refers to weight average molecular weight.

As used herein, “high molecular weight” is a molecular weight of greater than 1,250,000 Da, “low molecular weight” is a molecular weight less than 250,000 Da, and “intermediate molecular weight” is a molecular weight ranging from 250,000 Da to 1,250,000 Da.

Hyaluronic acid within a given composition is said to have “high average molecular weight” when the average molecular weight of the hyaluronic acid corresponds to a high molecular weight (defined above). Hyaluronic acid within a given composition is said to display a “high peak molecular weight” when it exhibits a peak molecular weight that corresponds to a high molecular weight (defined above). As used herein, “high molecular weight hyaluronic acid” includes hyaluronic acid that has a high average molecular weight, hyaluronic acid that has a mono-modal distribution and a high peak molecular weight, and hyaluronic acid in which 80 wt % of the hyaluronic acid molecules have a high molecular weight.

Hyaluronic acid within a given composition is said to have “low average molecular weight” when the average molecular weight of the hyaluronic acid corresponds to a low molecular weight (defined above). Hyaluronic acid within a given composition is said to display a “low peak molecular weight” when it exhibits a peak molecular weight that corresponds to a low molecular weight (defined above). As used herein, “low molecular weight hyaluronic acid” includes hyaluronic acid that has a low average molecular weight, hyaluronic acid that has a mono-modal distribution and a low peak molecular weight, and hyaluronic acid in which 80 wt % of the hyaluronic acid molecules have a low molecular weight.

Hyaluronic acid within a given composition is said to have “intermediate average molecular weight” when the average molecular weight of the hyaluronic acid corresponds to an intermediate molecular weight (defined above). Hyaluronic acid within a given composition is said to display an “intermediate peak molecular weight” when it exhibits a peak molecular weight that corresponds to an intermediate molecular weight (defined above). As used herein, “intermediate molecular weight hyaluronic acid” includes hyaluronic acid that has an intermediate average molecular weight, hyaluronic acid that has a mono-modal distribution and an intermediate peak molecular weight, and hyaluronic acid in which 80 wt % of the hyaluronic acid molecules have an intermediate molecular weight.

Low molecular weight hyaluronic acid, where present, may provide shorter term release for immediate action, intermediate molecular weight hyaluronic acid, where present, may provide intermediate term release, and high molecular weight hyaluronic acid, where present, may provide longer term release for extended action.

In certain embodiments, a single type of hyaluronic acid releasing composition, for instance, a single type of liquid, semi-liquid or solid composition (e.g., a single type of layer, particle, viscous liquid, gel, etc.) is employed in the devices of the invention.

Where a single type of hyaluronic acid releasing composition is employed, the average molecular weight of the hyaluronic acid within the composition may fall within any of the above molecular weight ranges. In some embodiments, hyaluronic acid in the composition may have a high average molecular weight (defined above), a low average molecular weight (defined above), or an intermediate average molecular weight (defined above), preferably a high average molecular weight.

Where a single type of hyaluronic acid releasing composition is employed, the hyaluronic acid within the composition may have a mono-modal distribution with a peak molecular weight that falls within any of the preceding molecular weight ranges. In some embodiments, hyaluronic acid within the composition may have a mono-modal distribution with a high peak molecular weight (defined above), a low peak molecular weight (defined above), or an intermediate peak molecular weight (defined above), preferably a high peak molecular weight.

Where a single type of hyaluronic acid releasing composition is employed, the hyaluronic acid within the composition may have a multi-modal distribution. For example, the hyaluronic acid within the composition may have a bi-modal distribution with two peak molecular weights within any of the preceding molecular weight ranges. For instance, the hyaluronic acid may have a first peak molecular weight that corresponds to a high peak molecular weight and a second peak molecular weight that corresponds to a low peak molecular weight, or the hyaluronic acid may have a first peak molecular weight that corresponds to a high peak molecular weight and a second peak molecular weight that corresponds to an intermediate peak molecular weight. As another example, the hyaluronic acid within the composition may have a tri-modal distribution with three peak molecular weights within any of the preceding molecular weight ranges. For instance, the hyaluronic acid may have a first peak molecular weight that corresponds to a high peak molecular weight, a second peak molecular weight that corresponds to a low peak molecular weight and a third peak molecular weight that corresponds to an intermediate peak molecular weight.

Where a single type of hyaluronic acid releasing composition is employed, in certain embodiments, the hyaluronic acid within the composition may have a molecular weight distribution such that 10 to 30 wt % of the hyaluronic acid has a low molecular weight, 40 to 70 wt % of the hyaluronic acid has an intermediate molecular weight, and 20 to 50 wt % of the hyaluronic acid has a high molecular weight. To form such a material, two or more batches of hyaluronic acid may be blended before loading on/in to the device.

In certain embodiments, a multiple types of hyaluronic acid releasing compositions, for instance, multiple types of liquid, semi-liquid or solid compositions (e.g., multiple types of layers, particles, viscous liquids, gels, etc.) are employed in the devices of the invention.

Where multiple types of hyaluronic acid releasing compositions are employed, in some embodiments, the devices comprise: a first composition comprising low molecular weight hyaluronic acid (defined above) and a second composition comprising high molecular weight hyaluronic acid (defined above).). In some embodiments, the devices comprise: a first composition comprising high molecular weight hyaluronic acid and a second composition comprising intermediate molecular weight hyaluronic acid (defined above). In some embodiments, the devices comprise: a first composition comprising low molecular weight hyaluronic acid, a second composition comprising intermediate molecular weight hyaluronic acid, and a third composition comprising high molecular weight hyaluronic acid (defined above).

In certain embodiments, the medical devices of the invention comprise a single layer of a hyaluronic acid releasing composition. In certain other embodiments, the medical devices may comprise two, three or more coating layers of the same or differing hyaluronic acid releasing composition, either disposed beside one another or disposed on top of each other.

For example, the medical device may comprise inner and outer coating layers and wherein the inner layer comprises high molecular weight hyaluronic acid and the outer layer comprises either low molecular weight hyaluronic acid or intermediate molecular weight hyaluronic acid. As another example, the medical device may comprise inner, intermediate and outer coating layers in which the inner layer comprises high molecular weight hyaluronic acid, the middle layer comprises intermediate molecular weight hyaluronic acid and the outer layer comprises low molecular weight hyaluronic acid.

In certain embodiments, hyaluronic acid releasing compositions may be provided in non-layer form, for instance, in a form suitable for providing the compositions in an internal or external reservoir (e.g. in viscous liquid form, gel form, particle form, rod form, etc.). In some of these embodiments, such devices may be provided with a first hyaluronic acid releasing composition (e.g., a first viscous liquid, gel, rod, particle population, etc.) which comprises high molecular weight hyaluronic acid and a second hyaluronic acid releasing composition (e.g., a second viscous liquid, gel, rod, particle population, etc.) which comprises either low molecular weight hyaluronic acid or intermediate molecular weight hyaluronic acid. In some embodiments, such devices may be provided with a first hyaluronic acid releasing composition which comprises high molecular weight hyaluronic acid, a second hyaluronic acid releasing composition which comprises intermediate molecular weight hyaluronic acid, and a third hyaluronic acid releasing composition which comprises low molecular weight hyaluronic acid.

As previously indicated, in some embodiments, the medical devices of the invention comprise one or more reservoirs. The one or more reservoirs may be, for example, formed in the surface of a substrate or may be internal to the medial device with one or more passageways extending from the reservoir to the surface of the device to allow elution of the hyaluronic acid from within the device.

Examples of reservoirs formed in the surface of a substrate surface (also referred to herein as “surface reservoirs”) include depressions such as trenches, blind holes and pores, among others. Surface reservoirs may be created in a great variety of shapes and sizes. Multiple surface reservoirs can be provided in a near infinite variety of arrays. Examples of blind holes include those whose lateral dimensions at the surface are circular, polygonal (e.g., triangular, quadrilateral, penta-lateral, etc.), as well as blind holes of various other regular and irregular shapes and sizes. Trenches include simple linear trenches, wavy trenches, trenches formed from linear segments whose direction undergoes an angular change (e.g., zigzag trenches), and linear trench networks intersecting various angles, as well as other regular and irregular trench configurations. The surface reservoirs can be of any suitable size. For example, the medical devices of the invention typically contain surface reservoirs whose smallest lateral dimension (e.g., the width) is less than 500 um (micrometers), for example, ranging from 500 micrometers to 200 micrometers to 100 micrometers to 10 micrometers to 1 micrometer or less.

In other embodiments, the medical device comprises one or more internal reservoirs that are internal to the medial device with one or more passageways extending from the reservoir to the surface of the device.

For example, the medical device may comprise a substrate with an internal lumen (which may be in addition to one or more other lumens, such as drainage lumens, working channels, etc.) with access to the exterior of the device (e.g., via one or more pores, holes, slots, etc.). One or more larger holes with plugs may also be provided, for example, in order to load the device with one or more types of hyaluronic acid releasing compositions (e.g., liquids, gels, particles, rods, etc.). Such internal lumens may extend partially or entirely through the device. Where the lumen extends entirely through the device, plugs may be provided at each end.

As indicated above, the medical devices of the invention are urological medical devices, including any medical device that is suitable for placement in the urinary tract of a subject, including the kidneys (e.g., in the renal calyx, renal pelvis, etc.), ureters, bladder and urethra. These include elongated devices, including elongated devices having any of a variety of solid and hollow cross-sections (e.g., single lumen, multi-lumen, rod-shaped devices) including those with overall cross-sections that are circular, oval, triangular, and rectangular in shape, among many other regular and irregular overall cross-sections. Specific examples include urological stents, for example, urethral and ureteral stents, urological catheters (e.g., drainage catheters, guide catheters, etc.), guidewires, urological scopes (e.g., cytoscopes, ureteroscopes, nephroscopes, etc.), tissue engineering scaffolds, grafts and patches, among others.

In some embodiments, devices may be employed that take on a particular beneficial shape in vivo, for example, immediately upon removal of a guide wire or emergence from a channel (e.g., due to elastic rebound of the material) or upon application of an external stimulus such as heat or light (e.g., where a shape memory material such as a shape memory polymer is employed). For example, the device may take on a non-linear form such as a coiled configuration. Such constructions allow the medical device to be held in place in the urinary tract, for example, by forming a coil or other retention element in the kidney (e.g., in the renal calyx and/or renal pelvis), the bladder, or both. In other embodiments, the devices may comprise a balloon element that can be inflated to hold the device in place.

Several embodiments will now be described with regard to ureteral stents, although it should be understood that the invention is not so limited.

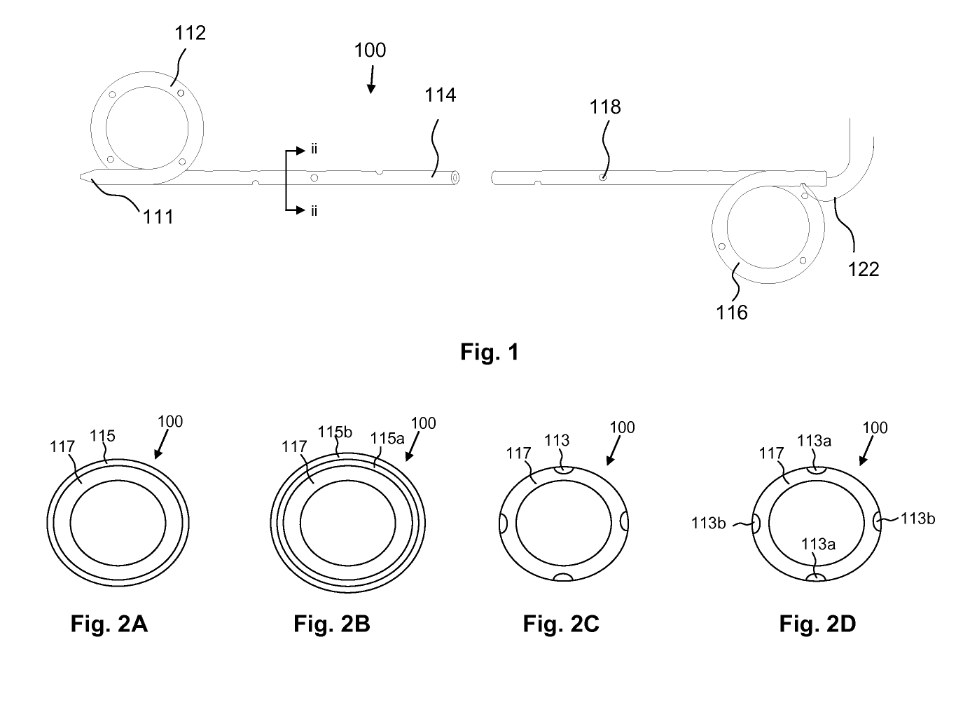

A schematic illustration of a ureteral stent in accordance with an embodiment of the invention is shown in FIG. 1. The stent 100 is a tubular polymeric extrusion which includes a renal coil 112, a shaft 114 and a bladder coil 116. Polymeric materials for forming the tubular polymeric extrusion include polyurethane and poly(ethylene-co-vinyl acetate) (EVA), among others. Among EVA copolymers are included random and other copolymers having a vinyl acetate weight percent ratio of from about 0.5% to 1% to 2% to 5% to 15% to 20% to 30% to 40% or more. In general, the higher the vinyl acetate content, the lower the stiffness and Durometer of the EVA. A tubular polymeric extrusion may be produced having distinct end regions of different Durometer value with a transitional region in between.

The stent 100 shown is further provided with the following: (a) a tapered tip 111, to aid insertion, (b) multiple side ports 118 (one numbered), which are arranged in a spiral pattern down the length of the body, to promote drainage, and (c) a nylon suture 122, which aids in positioning and withdrawal of the stent, as is known in that art. During placement, such ureteral stents 100 are commonly placed over a urology guide wire, through a cytoscope and advanced into position with a positioner. Once the proximal end of the stent is advanced into the kidney/renal calyx, the guide wire is removed, allowing coils 112, 116 to form in the kidney and bladder.

Three different cross sections taken along plane ii-ii in FIG. 1 are shown in FIGS. 2A-2C.

In accordance with one embodiment, shown in FIG. 2A, the ureteral stent 100 includes a tubular polymeric extrusion 117 coated with a single layer 115 of a hyaluronic acid releasing composition. For example, in one embodiment, the layer 115 may comprise high molecular weight hyaluronic acid. In another embodiment, the layer 115 may comprise hyaluronic acid with a multi-modal distribution that displays a high peak molecular weight and a low and/or an intermediate peak molecular weight. In another embodiment, the layer 115 varies in composition along the length of the ureteral stent for example, the layer 115 may comprise high molecular weight hyaluronic acid at the renal coil 112 and low or intermediate molecular weight hyaluronic acid at the bladder coil 116, with a stepwise or gradual transition between the two coils. In some embodiments, the layer 115 may comprise high molecular weight hyaluronic acid at the renal coil 112, intermediate weight hyaluronic acid in center of the shaft and low molecular weight hyaluronic acid at the bladder coil 116, with a stepwise or gradual transition between the two coils.

In accordance with another embodiment, shown in FIG. 2B, the ureteral stent 100 includes a tubular polymeric extrusion 117 coated with an inner layer 115a of a first hyaluronic acid releasing composition and an outer layer 115b of a second hyaluronic acid releasing composition that differs from the first composition. For example, in one embodiment, the inner layer 115a may comprise high molecular weight hyaluronic acid and the outer layer 115a may comprise either low or intermediate molecular weight hyaluronic acid.

In accordance with another embodiment, shown in FIG. 2C, the ureteral stent 100 includes a tubular polymeric extrusion 117 having surface reservoirs (four shown), for instance, trenches or blind holes, that are filled with a hyaluronic acid releasing composition 113. For example, in one embodiment, the composition 113 may comprise high molecular weight hyaluronic acid (defined above). In another embodiment, the composition 113 may comprise hyaluronic acid with a multi-modal distribution that displays a high peak molecular weight and a low and/or intermediate peak molecular weight (defined above). In another embodiment, the composition 113 varies in composition along the length of the ureteral stent; for example, the composition 113 may comprise high molecular weight hyaluronic acid at the renal coil 112 and low or intermediate molecular weight hyaluronic acid at the bladder coil 116, with a stepwise or gradual transition between the two coils. In some embodiments, the composition 113 may comprise high molecular weight hyaluronic acid at the renal coil 112, intermediate weight hyaluronic acid in center of the shaft and low molecular weight hyaluronic acid at the bladder coil 116, with a stepwise or gradual transition between the two coils.

In accordance with yet another embodiment, shown in FIG. 2D, the ureteral stent 100 includes a tubular polymeric extrusion 117 having surface reservoirs (four shown), for instance, trenches or blind holes, filled with a first hyaluronic acid releasing composition 113a and a second hyaluronic acid releasing composition 113b that differs from the first composition 113a. For example, in one embodiment, the first hyaluronic acid releasing composition 113a may comprise high molecular weight hyaluronic acid and the second hyaluronic acid releasing composition 113b may comprise low molecular weight hyaluronic acid.

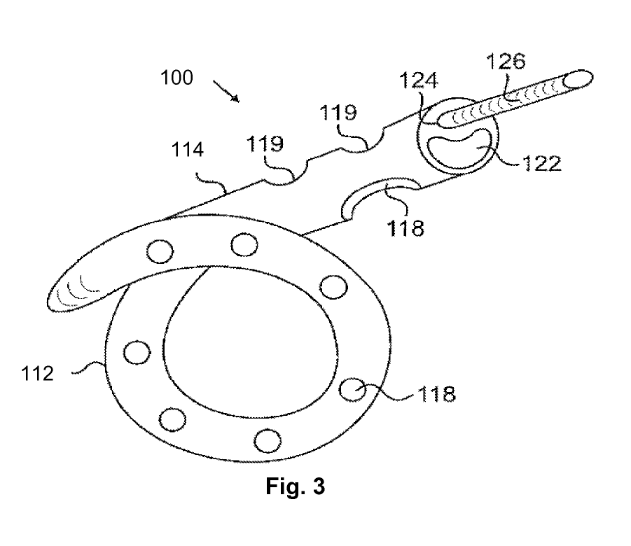

Another embodiment of the invention is schematically illustrated in FIG. 3, which shows a partial cross-sectional view of a ureteral stent 100 in accordance with an embodiment of the invention. As in FIG. 1, FIG. 3 illustrates a renal coil 112 and a portion of the shaft 114 extending in the direction of the bladder coil (not shown) of the stent 100. The shaft 114 is a dual lumen design, comprising a drainage lumen 122 with drainage ports 118 to promote drainage, and a hyaluronic acid delivery lumen 124 with delivery ports 119 to promote hyaluronic acid delivery. Within the hyaluronic acid delivery lumen 124 is disposed a solid cylindrical hyaluronic acid releasing composition 126 (i.e., a rod). For illustration purposes, the cross-section for the hyaluronic acid releasing composition 126 is taken at a position more proximal (bladder end) than the cross-section taken for the shaft 112 as shown. The hyaluronic acid releasing composition 126 may be introduced into the delivery lumen 124 from either end of the stent 110.

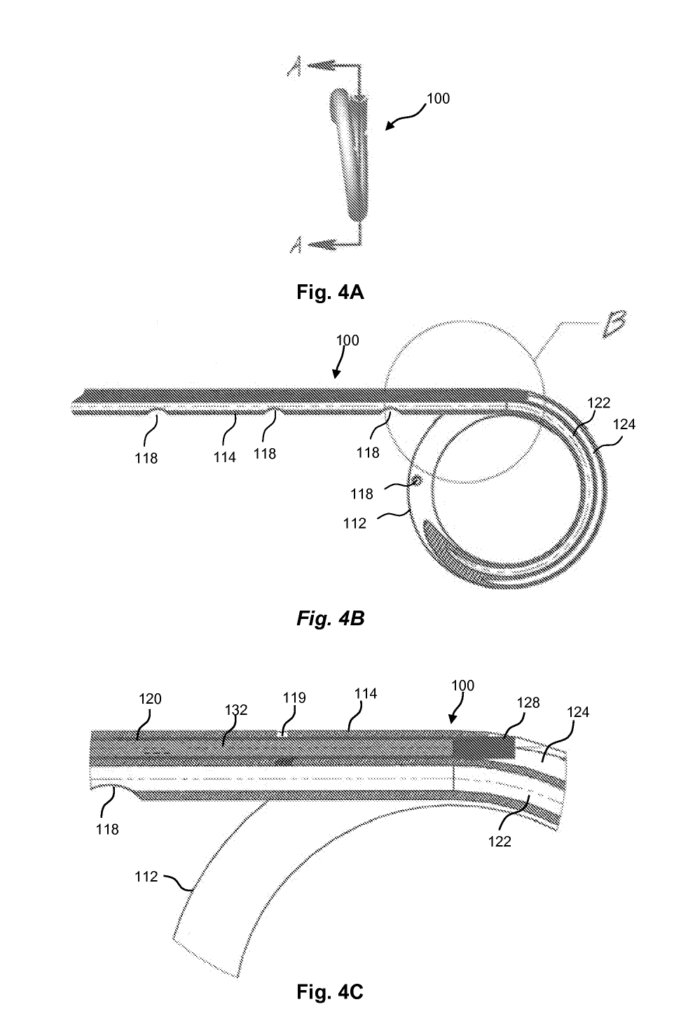

Turning now to FIG. 4A, there is shown a schematic end view of a ureteral stent 100 in accordance with an embodiment of the invention. FIG. 4B is a partial cross-sectional view of the stent 100 of FIG. 4A, taken along plane A-A. FIG. 4C is an expanded view corresponding to region B in FIG. 4B. FIGS. 4A-4C illustrate the renal coil 112 and a portion of the shaft 114 extending in the direction of the bladder coil (not shown) of the stent 100. The shaft 114 is a dual lumen design, comprising a drainage lumen 122 with drainage ports 118 to promote urine drainage, and a delivery lumen 124 with hyaluronic acid delivery ports 119 to promote hyaluronic acid delivery. The shaft 114 may comprise a polymer (e.g., EVA, polyurethane, etc.). The delivery lumen 124 of the shaft 114 is lined with a porous material 120. In a specific example, the porous material may be a porous material like those described in U.S. Pat. No. 5,282,785 to Shapland et al., U.S. Pat. No. 5,569,198 to Racchini, or U.S. Pat. No. 5,458,568 to Racchini et al. The delivery lumen 124 of the shaft 114 is provided with a removable plug 128 (or a septum) which allows a liquid solution or dispersion of a hyaluronic acid containing composition 132 to be introduced into the delivery lumen 124 and at least partially absorbed by the layer of porous material 120. Upon introduction of the stent 100 into the body of a patient, the drainage lumen 122 (with ports drainage ports 118) acts to promote drainage of urine through the ureter, whereas the delivery lumen 124 (with hyaluronic acid delivery ports 119) acts to promote hyaluronic acid delivery. For example, urine can enter the delivery lumen 124 via delivery ports 119, which urine takes up the hyaluronic acid and is then transported from the delivery lumen 124 to the patient via delivery ports 119.

Although the preceding embodiment describes a liquid hyaluronic acid containing composition 132 in association with a porous material 120, in other embodiments, the hyaluronic acid containing composition 132 may be in the form of a viscous liquid, gel, or solid (e.g., a rod, particles, etc.), in which case the porous material 120 may be dispensed with.

Various aspects of the invention of the invention relating to the above are enumerated in the following paragraphs:

Aspect 1. A urological medical device comprising a substrate and hyaluronic acid releasably disposed on or within the substrate, wherein upon implantation in a urological tract of a subject, the medical device releases the hyaluronic acid in an amount effective to cause a reduction in microbial infection.

Aspect 2. The urological medical device of aspect 1, wherein the reduction in microbial infection is sustained for at least 2 weeks.

Aspect 3. The urological medical device of aspect 1, wherein said device comprises a composition that comprises high molecular weight hyaluronic acid.

Aspect 4. The urological medical device of aspect 1, wherein said device comprises a composition that comprises hyaluronic acid with a multi-modal molecular weight distribution which displays a first peak molecular weight that corresponds to a high peak molecular weight and a second peak molecular weight that corresponds to a low or intermediate peak molecular weight.

Aspect 5. The urological medical device of aspect 1, wherein said device comprises a composition that comprises hyaluronic acid having a molecular weight distribution such that 10 to 30 wt % of the hyaluronic acid has a low molecular weight, 40 to 70 wt % of the hyaluronic acid has an intermediate molecular weight, and 20 to 50 wt % of the hyaluronic acid has a high molecular weight.

Aspect 6. The urological medical device of aspect 1, wherein said device comprises a first composition comprising low or intermediate molecular weight hyaluronic acid and a second composition comprising high molecular weight hyaluronic acid.

Aspect 7. The urological medical device of aspect 1, wherein said device comprises a first composition comprising low molecular weight hyaluronic acid, a second composition comprising high molecular weight hyaluronic acid, and a third composition comprising intermediate molecular weight hyaluronic acid.

Aspect 8. The urological medical device of aspect 1, wherein said medical device comprises a coating that comprises said hyaluronic acid.

Aspect 9. The urological medical device of aspect 8, wherein said medical device comprises a single coating layer that comprises high molecular weight hyaluronic acid.

Aspect 10. The urological medical device of aspect 8, wherein said medical device comprises a single coating layer that comprises hyaluronic acid having a molecular weight distribution such that 10 to 30 wt % of the hyaluronic acid has a low molecular weight, 40 to 70 wt % of the hyaluronic acid has an intermediate molecular weight, and 20 to 50 wt % of the hyaluronic acid has a high molecular weight.

Aspect 11. The urological medical device of aspect 8, wherein said medical device comprises a first coating layer comprising low or intermediate molecular weight hyaluronic acid disposed over a second coating layer comprising high molecular weight hyaluronic acid.

Aspect 12. The urological medical device of aspect 8, wherein said medical device comprises a first coating layer comprising low molecular weight hyaluronic acid disposed over a second coating layer comprising intermediate molecular weight hyaluronic acid disposed over and a third coating layer comprising high molecular weight hyaluronic acid.

Aspect 13. The urological medical device of aspect 1, wherein the medical device comprises a composition in the form of a gel that comprises hyaluronic acid and water.

Aspect 14. The urological medical device of aspect 1, comprising a surface reservoir that comprises said hyaluronic acid.

Aspect 15. The urological medical device of aspect 14, wherein said surface reservoir is selected from a hole and a trench.

Aspect 16. The urological medical device of aspect 1, comprising an internal reservoir that comprises said hyaluronic acid.

Aspect 17. The urological medical device of aspect 16, wherein said internal reservoir is in the form of an internal lumen with access to the exterior of the device via one or more openings.

Aspect 18. The urological medical device of aspect 1, wherein said urological medical device is a ureteral stent.

Aspect 19. The urological medical device of aspect 18, wherein said ureteral stent comprises an internal reservoir that is loaded with a solid composition that comprises said hyaluronic acid.

Aspect 20. The urological medical device of aspect 18, wherein said hyaluronic acid is provided at the surface of said device in the form of a gel.

Although various embodiments are specifically illustrated and described herein, it will be appreciated that modifications and variations of the present invention are covered by the above teachings and are within the purview of any appended claims without departing from the spirit and intended scope of the invention.

1. The urological medical device comprising a substrate and hyaluronic acid releasably disposed on or within the substrate, wherein upon implantation in a urological tract of a subject, the medical device releases the hyaluronic acid in an amount effective to cause a reduction in microbial infection.

2. The urological medical device of claim 1, wherein the reduction in microbial infection is sustained for at least 2 weeks.

3. The urological medical device of claim 1, wherein said device comprises a composition that comprises high molecular weight hyaluronic acid.

4. The urological medical device of claim 1, wherein said device comprises a composition that comprises hyaluronic acid with a multi-modal molecular weight distribution which displays a first peak molecular weight that corresponds to a high peak molecular weight and a second peak molecular weight that corresponds to a low or intermediate peak molecular weight.

5. The urological medical device of claim 1, wherein said device comprises a composition that comprises hyaluronic acid having a molecular weight distribution such that 10 to 30 wt % of the hyaluronic acid has a low molecular weight, 40 to 70 wt % of the hyaluronic acid has an intermediate molecular weight, and 20 to 50 wt % of the hyaluronic acid has a high molecular weight.

6. The urological medical device of claim 1, wherein said device comprises a first composition comprising low or intermediate molecular weight hyaluronic acid and a second composition comprising high molecular weight hyaluronic acid.

7. The urological medical device of claim 1, wherein said device comprises a first composition comprising low molecular weight hyaluronic acid, a second composition comprising high molecular weight hyaluronic acid, and a third composition comprising intermediate molecular weight hyaluronic acid.

8. The urological medical device of claim 1, wherein said medical device comprises a coating that comprises said hyaluronic acid.

9. The urological medical device of claim 8, wherein said medical device comprises a single coating layer that comprises high molecular weight hyaluronic acid.

10. The urological medical device of claim 8, wherein said medical device comprises a single coating layer that comprises hyaluronic acid having a molecular weight distribution such that 10 to 30 wt % of the hyaluronic acid has a low molecular weight, 40 to 70 wt % of the hyaluronic acid has an intermediate molecular weight, and 20 to 50 wt % of the hyaluronic acid has a high molecular weight.

11. The urological medical device of claim 8, wherein said medical device comprises a first coating layer comprising low or intermediate molecular weight hyaluronic acid disposed over a second coating layer comprising high molecular weight hyaluronic acid.

12. The urological medical device of claim 8, wherein said medical device comprises a first coating layer comprising low molecular weight hyaluronic acid disposed over a second coating layer comprising intermediate molecular weight hyaluronic acid disposed over and a third coating layer comprising high molecular weight hyaluronic acid.

13. The urological medical device of claim 1, wherein the medical device comprises a composition in the form of a gel that comprises hyaluronic acid and water.

14. The urological medical device of claim 1, comprising a surface reservoir that comprises said hyaluronic acid.

15. The urological medical device of claim 14, wherein said surface reservoir is selected from a hole and a trench.

16. The urological medical device of claim 1, comprising an internal reservoir that comprises said hyaluronic acid.

17. The urological medical device of claim 16, wherein said internal reservoir is in the form of an internal lumen with access to the exterior of the device via one or more openings.

18. The urological medical device of claim 1, wherein said urological medical device is a ureteral stent.

19. The urological medical device of claim 18, wherein said ureteral stent comprises an internal reservoir that is loaded with a solid composition that comprises said hyaluronic acid.

20. The urological medical device of claim 18, wherein said hyaluronic acid is provided at the surface of said device in the form of a gel.
